(19) United States
(12) Patent Application Publication
(10) Pub. No.: US 2025/0384508 A1
(43) Pub. Date: Dec. 18, 2025

(54) SYSTEMS AND METHODS FOR REVIEWING AND ANALYZING INFORMATION SUBJECT TO ENFORCEMENT OR REGULATION

(71) Applicant: Pharmasift, LLC, Saddle River, NJ (US)

(72) Inventor: Zafar TOOR, Saddle River, NJ (US)

(21) Appl. No.: 19/188,405

(22) Filed: Apr. 24, 2025

Related U.S. Application Data
(63) Continuation of application No. 18/982,528, filed on Dec. 16, 2024.
(60) Provisional application No. 63/660,128, filed on Jun. 14, 2024.

(51) Int. Cl. G06Q 50/26; G06Q 30/0601

(52) U.S. Cl. CPC G06Q 50/26; G06Q 30/0277; G16H 70/40

(57) ABSTRACT

The present disclosure is directed to systems and methods for reviewing and analyzing information related to products, services, or industries subject to regulation to check whether the information is in compliance or may be subject to enforcement action. In one embodiment, a computer-implemented method includes training a machine learning (ML) algorithm with a selected set of materials that includes a proprietary portion and a public portion; reviewing at least one material by the ML algorithm trained with the selected set of materials; and outputting at least one result of the reviewing by the ML algorithm.

### PRIORITY CLAIM

This application is a continuation of U.S. patent application Ser. No. 18/982,528, filed on Dec. 16, 2024, which claims priority to U.S. Provisional Patent Application No. 63/660,128, filed Jun. 14, 2024, the entireties of both of which are incorporated herein by reference.

### TECHNICAL FIELD

Embodiments of this disclosure relate generally to the field of computing systems and more particularly to using specially trained artificial intelligence to review and analyze information that is subject to enforcement or regulation.

### BACKGROUND

Individuals and organizations working in and with regulated products and industries must comply with applicable government rules and regulations. These rules and regulations can be numerous, complex, and subject to change. Therefore, it can be difficult to stay abreast of what is relevant with respect to any particular product or activity and ensure compliance without triggering negative government feedback or action.

For example, the U.S. Food & Drug Administration's (FDA) Office of Prescription Drug Promotion (OPDP) regulates communications related to drugs and pharmaceuticals. Such communications must be fair and accurate with respect to medical, legal, and regulatory content, and if any is found to be misleading or untruthful a warning or enforcement action may occur. If this happens, the process can become (even more) protracted and expensive.

Therefore, drug companies—and others working in or with regulated products and industries—take steps to ensure compliance. But existing steps can be expensive and time-consuming, and given the nature of regulated industries it can be difficult to stay up to date on the latest requirements and guidance.

### SUMMARY

A need exists, therefore, for systems and methods for reviewing and analyzing information related to products, services, or industries subject to regulation to check whether the information is in compliance or may be subject to enforcement action.

In one embodiment, an artificial intelligence (AI)-powered regulatory review system comprises a large language model (LLM) trained on a data set comprising at least one of life sciences-related advertising and promotion rules or regulations, wherein at least a portion of the data set is proprietary and not publicly available; wherein the LLM is configured to receive proposed regulated content and generate a compliance analysis of the received proposed regulate content based on the data set and at least one jurisdiction-specific regulatory framework.

In another embodiment, a method for assisting in the review and approval of life sciences content comprises receiving proposed life sciences content; parsing the received proposed life sciences content to identify any of claims, references, or risk-benefit language; comparing the identified claims, references, or risk-benefit language to a database of at least one of jurisdiction-specific life sciences rules or regulations; and generating suggested modifications to the proposed life sciences content to enhance compliance with the at least one of jurisdiction-specific life sciences rules or regulations.

In yet another embodiment, a conversational AI system for regulatory guidance, comprises a large language model (LLM) trained on a data set comprising at least one of life sciences advertising and promotion rules or regulations, wherein at least a portion of the data set is proprietary and not publicly available; a user interface configured to: receive a user-submitted query about a life sciences claim, and provide a response from the LLM to the user-submitted query referencing at least one of specific regulatory guidance documents, precedent enforcement actions, and product-specific labeling, or proprietary rules based on the data set.

In a further embodiment, an integrated system comprises a large language model (LLM) trained on a data set comprising life sciences advertising and promotion regulations, wherein at least a portion of the data set is proprietary and not publicly available; and a user interface configured to: receive a user-submitted query comprising any of a natural language question about regulated content or a related claim, proposed regulated content as text, or proposed regulated content as a file, and provide a response from the LLM to the user-submitted query referencing at least one of specific regulatory guidance documents, precedent enforcement actions, product-specific labeling, and proprietary rules based on the data set.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

This disclosure may be more completely understood in consideration of the following description of various embodiments in connection with the accompanying figures, in which:

FIG. 1 is a conceptual block diagram of a computer system according to an embodiment.

FIG. 2A is a flowchart according to an embodiment.

FIG. 2B is a screenshot of an input user interface according to an embodiment.

FIG. 2C is a screenshot of an output user interface according to an embodiment.

FIG. 3A is a screenshot of an output user interface according to an embodiment.

FIG. 3B is a screenshot of an output user interface according to an embodiment.

FIG. 3C is a screenshot of an output user interface according to an embodiment.

FIG. 4A is a screenshot of an input user interface according to an embodiment.

FIG. 4B is a screenshot of an input user interface according to an embodiment.

FIG. 4C is a screenshot of an output user interface according to an embodiment.

FIG. 4D is a screenshot of an input user interface according to an embodiment.

FIG. 4E is a screenshot of an output user interface according to an embodiment.

FIG. 5 is a functional diagram of a chat agent system according to an embodiment.

FIG. 6A is a functional diagram of an AI model selection feature of the chat agent system of FIG. 5 according to an embodiment.

FIG. 6B is a screenshot of a chat agent user interface according to an embodiment.

FIG. 6C is a functional diagram of a chat agent persona selection feature according to an embodiment.

While various embodiments are amenable to various modifications and alternative forms, specifics thereof have been shown by way of example in the drawings and will be described in detail. It should be understood, however, that the intention is not to limit the disclosure or claims to the particular embodiments described. On the contrary, the intention is to cover all modifications, equivalents, and alternatives falling within the spirit and scope of the subject matter as defined by the claims.

### DETAILED DESCRIPTION

The present disclosure is directed to systems and methods for reviewing and analyzing information related to products, services, or industries subject to regulation to check whether the information is in compliance or may be subject to enforcement action. In various embodiments discussed herein, the systems include (and the methods implement) one or more specially trained machine learning (ML) algorithms. The special training can include feeding the ML algorithm with training data that includes proprietary information including or related to legal analysis, guideline interpretation, industry guidance, and other specially developed information, as well as selective publicly available information such as government laws or rules, enforcement action letters or other materials, approved information, and other information that is or could be relevant to the review and analysis of the subject information.

ML is a field of artificial intelligence (AI) in which algorithms can be trained by and learn from data in order to generalize new or unseen data. ML can be or include a large language model (LLM), which is an AI model (in particular, an artificial neural network, or ANN) that can understand and generate language. Conventional examples of LLMs include GPT, GEMINI, LLAMA, CLAUDE, and others. While examples discussed herein may use or apply ML and LLMs, other types of AI may be used in embodiments of the systems and methods of this disclosure without being limited by any particular mention or example related to ML used herein. Furthermore, examples or types of ML or AI or future iterations of these technologies and techniques not yet known can be relevant to or used with embodiments of this disclosure, particularly given the rapid pace of advancement of AI and ML at this time and the foundational understanding those of ordinary skill in the art will have with respect to these techniques as they evolve.

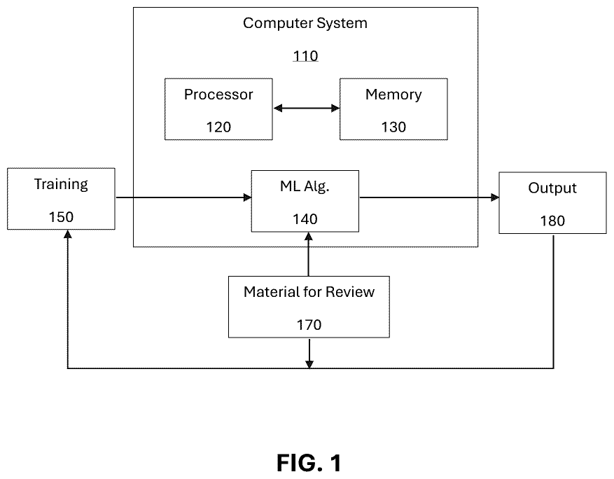

With reference to FIG. 1, ML algorithms and other AI techniques operate on, or are implemented at least in part by, a computer system 110. Computer system 110 can have virtually any suitable form, such as mainframe, mini, micro, super, server, or any combination of these or other types of computer systems. Moreover, computer system 110 can be virtual, cloud-based, or physical. In other words, the particular implementation of computer system 110 can vary.

In the simplified conceptual view of FIG. 1, computer system 110 comprises at least one processor 120 and memory 130. In other embodiments, the systems, methods, and algorithms discussed herein can operate in conjunction with at least one processor and memory. For example, in one embodiment processor 120 and memory 130 can be cloud-based, with user interaction facilitated via the internet, an application programming interface (API) presented on a computing device, such as a computer, laptop, smart phone, tablet, smart watch or other wearable, or other device or system, or some other computing device portal.

Processor 120 or any of the other systems or components discussed herein with respect to computer system 110 can be or work in cooperation with any programmable device (or system or network of devices) that accepts digital data as input, is configured to process the input according to instructions or algorithms, and provides results as outputs. In one example embodiment, processor 120 can be a central processing unit (CPU) or a microcontroller or microprocessor (or group of microcontrollers or microprocessors) configured to carry out the instructions of a computer program or software. Processor 120 is therefore configured to perform at least basic arithmetical, logical, and input/output operations.

Processor 120 includes or is communicatively coupled with memory 130 or other digital storage, which can comprise volatile or non-volatile memory as required by processor 120 to not only provide space to execute the instructions or algorithms, but also to provide the space to store the instructions themselves. In various embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, or optical disc storage, for example.

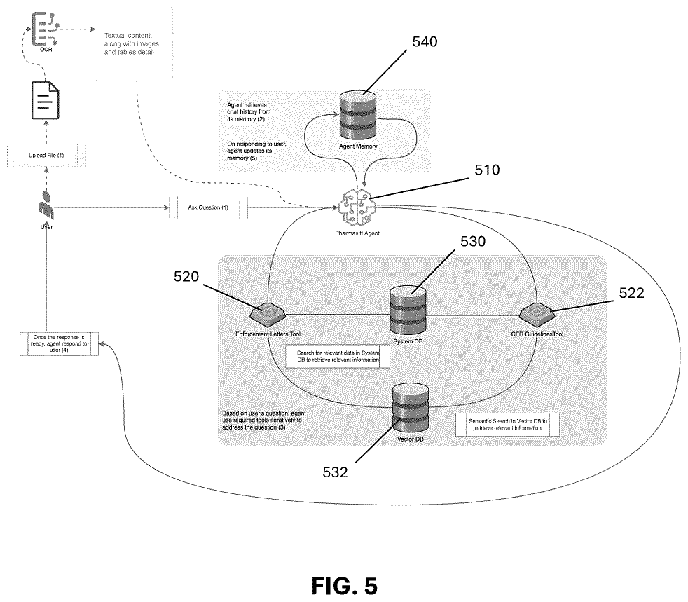

In some embodiments, the system of FIG. 1 (whether as part of memory 130, in communication with memory 130, or separately (e.g., in the cloud) includes or interacts with various databases. These databases can include one or more system databases, SQL databases, vector databases, curated or proprietary databases, data management systems, or other data stores or repositories. Specific examples relevant to some or all embodiments will be included herein (see, e.g., FIG. 5 and the related discussion).

The foregoing examples in no way limit the types of processing hardware or systems, or memory hardware or systems, that can be used in various embodiments, as these examples are given only by way of example and are not intended to limit the scope of the present disclosure or embodiments discussed herein. For example, both processor 120 and memory 130 can be cloud-based but nevertheless comprise physical infrastructure on a server or server farm.

Computer system 110 further comprises a machine learning (ML) algorithm 140. Though M L algorithm 140 is depicted separately in FIG. 1, those skilled in the art will recognize that ML algorithm 140 can be stored in memory 130 and executed by processor 120 in use and operation. In other embodiments, ML algorithm 140 can be stored remotely from computer system 110 or executed in conjunction with processors or memory separate from computer system 110. As such, FIG. 1 can be viewed as a functional or conceptual diagram, rather than a strict structural representation.

A base requirement for ML algorithm 140 is that it is an algorithm that can be trained by and learn from data in order to generalize new or unseen data. Though described as a machine learning algorithm, ML algorithm 140 can be any type of artificial intelligence (AI) or neural network (NN) algorithm.

In various embodiments, ML algorithm 140 receives training data and information (“training”) 150. As depicted in FIG. 1, training 150 is received from outside of computer system 110, but in other embodiments training 150 can be stored by memory 130 and provided therefrom to ML algorithm 140, or provided to ML algorithm 140 in some other way.

According to embodiments of this disclosure, training 150 comprises proprietary data and information specially developed with respect to a particular industry. For example, the proprietary data and information of training 150 can relate to life sciences industries, such as drugs and pharmaceuticals, medical devices, and related technologies, which typically are heavily regulated. In this example, the proprietary data and information of training 150 includes one or more of: professionally developed or reviewed legal analysis; interpretation of law, rules, or administrative guidelines; industry guidance based on actual experience or feedback; or any other specially developed information. In this way, ML algorithm 140 is trained with information as part of training 150 that is not generally publicly available, cannot be generated by a computer alone, and may be expertly produced or assembled with a goal of maximizing the effectiveness of ML algorithm 140 with respect to the relevant industry. Furthermore, training ML algorithm 140 with proprietary data and information separates and elevates the performance and efficacy of ML algorithm 140 as compared with conventional ML algorithms or AI systems that are commonly available via the internet but merely apply public information scrubbed from the internet.

In addition to specially selected and proprietary information, training 150 also can include selective publicly available information. Returning to the example related to life sciences industries including drugs and pharmaceuticals, the selective publicly available information can include government laws (e.g., U.S. Code, state laws, or local ordinances), rules (e.g., the U.S. Code of Federal Regulations, or CFR), enforcement action letters (e.g., those provided by the FDA OPDP or a state health or regulator entity), previously approved materials (e.g., advertisements, packaging, package inserts, promotional language, or images), non-binding final or draft guidance documents published or otherwise provided by regulatory bodies (such as the FDA, FTC, SEC, and others), and virtually any other information that is or could be relevant to the review and analysis of the subject information.

Regardless of the particular type or balance of proprietary and public information used in any specific example, a feature of some embodiments of this disclosure is that all of the sources of information, including those sources that are publicly available, are selected or reviewed for selection before or as part of being included in training 150. This ensures that any sources that may be prohibited, viewed as untrustworthy, or otherwise seen as problematic or troublesome are not considered by ML algorithm 140. This too separates and elevates the performance and efficacy of ML algorithm 140 as compared with conventional ML algorithms or AI systems that are commonly available via the internet but may factor in forums, sources, or data that are not considered reliable or valid.

In still other embodiments, filtering can be used with respect to training 150 or in other contexts. For example, selectively programmed or applied filters can be used based on the particular material for review 170 (or context thereof). In other examples, ML techniques can be used based on material for review 170, to process or filter data for training 150, or for some other purpose. Filtering can be helpful for several reasons, including selecting the most relevant data and information of training 150 to apply for a particular query or input, to identify discrepancies or conflicting information, or to attempt to analyze the efficacy or validity of information or sources. This can both improve the speed of ML algorithm 140 and improve the veracity of its results (e.g., content of report 180) with respect to the provided material for review 170.

Material for review 170 can take many forms. It can include written/textual documents or information as well as images, drawings, diagrams, videos, and other content. In the drug and pharmaceutical example, material for review 170 can comprise a print advertisement, a promotional material or communication, text for packaging or a package insert, a transcript for a commercial, a clip or video of a commercial, the content of a web page or site, non-promotional communications or materials such as disease state educational materials, or virtually any type of information that may be relevant to the sale, marketing, use, or production of a drug or pharmaceutical. The type of document can vary, such as PDF, DOC/DOCX, HTM/HTML, XML, JPG/JPEG, RTF, TXT, WAV, MPX, AAC, ZIP, PNG, AVI, MOV, FLV, PPT/PPTX, ODP, KEY, M 4A, and others, including those yet to be developed at the time of filing.

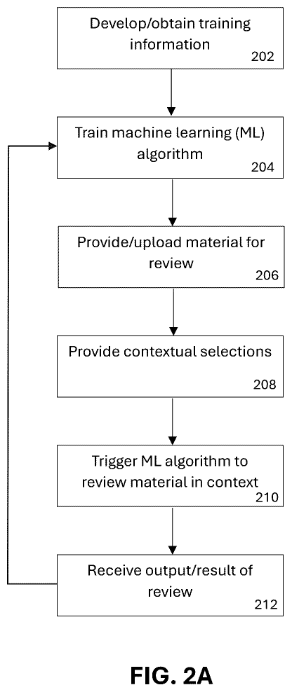

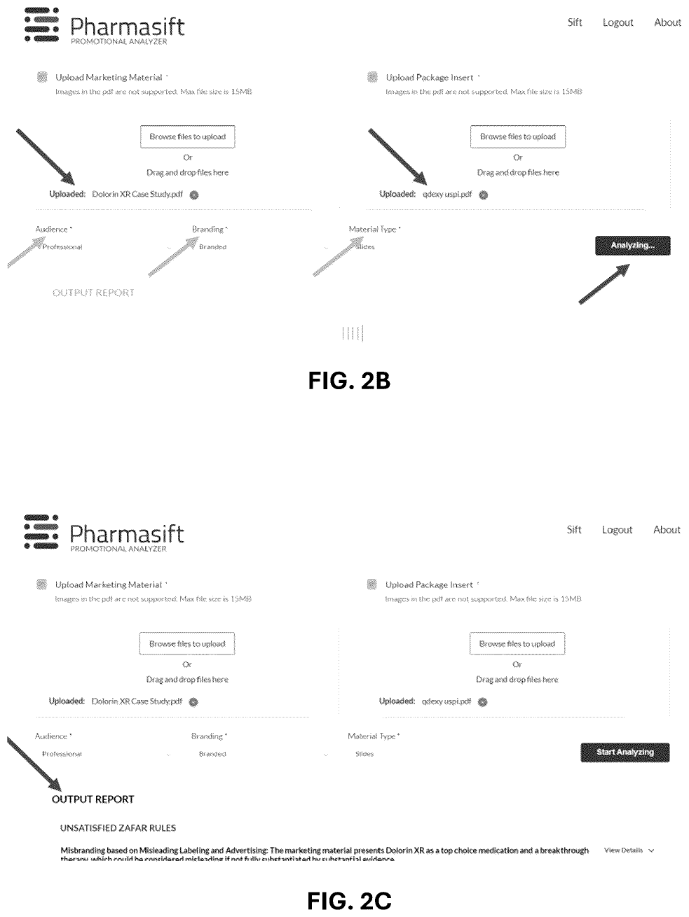

With this foundation, example operation will now be discussed with reference to FIG. 2A. The order of operations of FIG. 2A can vary from the particular depiction here. Additionally, in some embodiments one or more depicted operations may be omitted or duplicated, or additional operations not explicitly depicted in FIG. 2A may be included. For example, filtering can take place at one or more points but is not depicted in particular in FIG. 2A.

At 202, training data and information (e.g., training 150) is developed or otherwise obtained.

At 204, the training data and information is provided to the ML algorithm (e.g., ML algorithm 140), and the algorithm is trained based on the provided data and information. In some embodiments, the algorithm may already have been trained at some level, or based on some provided data and information, such that the training at 204 is additional or updated training.

At 206, the material desired to be reviewed (e.g., material for review 170) is uploaded or otherwise provided such that it is accessible to the algorithm. Material for review 170 can be a single document, a series of documents or files, or a package of many different types of materials. An example screenshot for this operation is depicted in FIG. 2B. The particular layout of and options included in FIG. 2B can vary in other embodiments.

In some embodiments, supplemental documents also can be provided to the ML algorithm. These can include documents and information that the ML algorithm can use as a reference in order to provide more customized and specific responses. In one embodiment, these documents can be specific to the user or the material to be reviewed, while in other embodiments the documents can be more generalized. In one way, these supplemental or additional documents can be considered to be additional training for the ML algorithm, or these documents can be considered to be contextual. Examples of supplemental documents include package inserts (prescribing information, full prescribing information, product information, FDA approved labeling) or references such as articles, reprints, clinical data, and the like.

Optionally, contextual selections related to the material for review can be provided for filtering, at 208. This is also shown in FIG. 2B. These selections can be made from dropdown menus as in FIG. 2B, via radio buttons, or in some other way and integrated with the provision/uploading at 206. In one example, categories of selections can include audience (e.g., professional, governmental, patient, or public), branding (e.g., branded or unbranded), and material type (e.g., document type or media type, such as text, slides, audio, video, or mixed/combination). Still other filter categories can be used in other embodiments, or some of these example categories can be omitted.

At 210, the ML algorithm is initiated or otherwise triggered to review the material provided, based on the current training and with any desired or relevant filtering applied. In the example of FIG. 2C, this would be accomplished by clicking on “Start analyzing . . . ,” though the particular language here can vary.

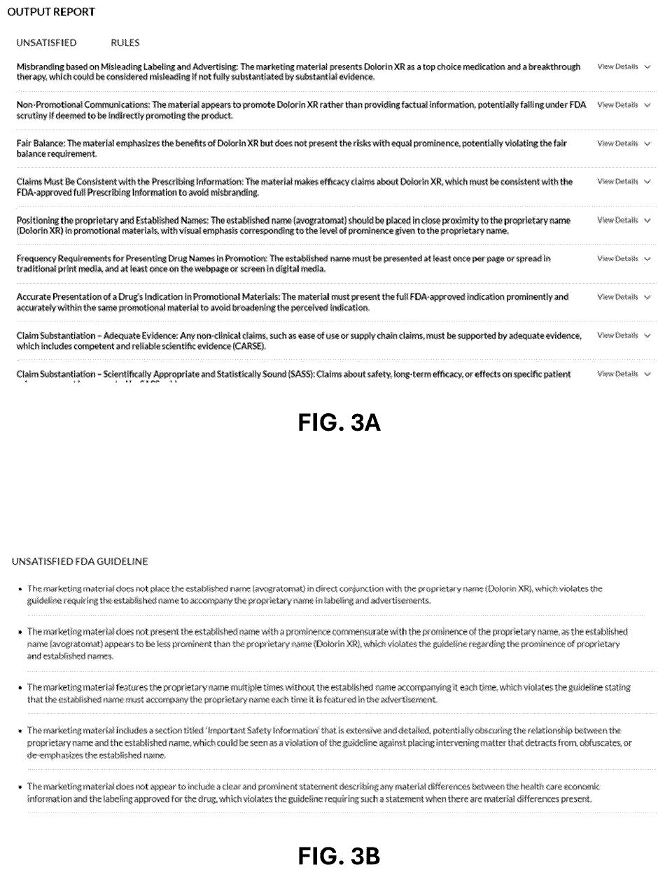

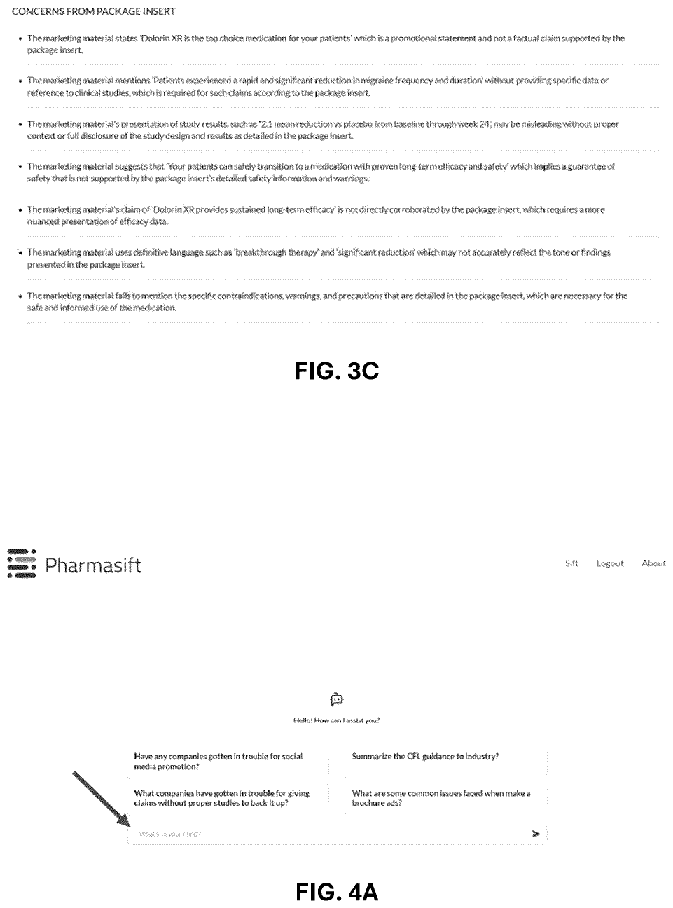

At 212, the result of the review is provided as output. One example of this is included in FIG. 2C (as well as FIGS. 3A, 3B, and 3C discussed below). This output can take many forms, including a written (i.e., generally electronic or digital, which can be reproduced in hardcopy) report, a marked up or annotated document, a bot-driven audio or video summary, or some other format.

A partial output report is included as an example in FIGS. 3A, 3B, and 3C. This example report relates to reviewed marketing material for a drug or pharmaceutical and includes three sections: Unsatisfied Rules (FIG. 3A), Unsatisfied FDA Guidelines (FIG. 3B), and Concerns from Package Insert (FIG. 3C).

Referring now to FIG. 3A, these concerns stem from review of the material with respect to the specially developed and selected proprietary review rules provided as part of training 150. In this example, these rules relate to aspects such as whether the material for review 170 is fair and balanced, not misbranded, fair and balanced, includes claims consistent with prescribing information, and more. An advantage of ML algorithm 140 and output 180 it provides is that problematic aspects or content can be explicitly identified and compared with the applicable rules or standards, enabling users to easily identify and address any issues.

In the example embodiment of FIG. 3A, a drop-down “View Details” option is provided, though this is optional and may be omitted, or its content may be included in the main report, in other embodiments. Here, additional information, such as quotes from material for review 170 or training 150, screenshots, annotated versions of material for review 170, or other content may be available. This additional information also can be presented in other ways or not included, in various embodiments. For example, some levels of details may be subscription based for users of the system, with corresponding details and information provided only to subscribed users.

Another portion of the report is included in FIG. 3B. Here, particular FDA guidelines that are not satisfied but are applicable to the drug and pharmaceutical example are identified in output 180.

The portion of the report in FIG. 3C relates to concerns about the package insert, which is included with pharmaceuticals. The first one is as follows: “The marketing material states ‘Dolorin XR is the top choice medication for your patients,’ which is a promotional statement and not a factual claim support by the package insert. Thus, this statement in the marketing materials was reviewed with respect to at least a related package insert, and ML algorithm 140 determined that this statement was promotional, not factual, and unsupported by the related package insert.

Another example from FIG. 3C is, “The marketing material fails to mention the specific contraindications, warnings, and precautions that are detailed in the packet insert, which are necessary for the safe and informed use of the medication.” Here ML algorithm 140 has identified content that is missing from the marketing material, a particularly sophisticated result (i.e., compared with simply analyzing the content that is present).

It can be seen by this example of output 180 that that ML algorithm 140 determined that various content and statements in material for review 150 would be problematic with respect to FDA OPDP and could trigger a warning or enforcement letter/action. The various content and statements can be specifically identified, along with the applicable rules, regulations, and standards, making it easy for a user to review and address identified issues.

Though not depicted in FIGS. 3A-3C, in some embodiments the problematic content can be annotated within the material for review 150 itself. This can make it even easier for issues to be found and corrected based on the review provided by ML algorithm 140. Still further, some embodiments can provide suggested corrections, edits, or missing content to add. Thus, ML algorithm 140 can both identify issues and provide suggested solutions.

Returning to FIGS. 1 and 2, in some embodiments output 180 is provided as training 150 to ML algorithm 140. In still other embodiments, material for review 150 is also provided as training 150 for ML algorithm 140. This enables ML algorithm 140 to continue to learn, develop, and advance based on different materials (170) and results (180), which can provide even better review and output 180 for other users.

Another aspect of ML algorithm 140 can be a chat, such as a so-called “chatbot,” feature. This feature can be used with and complimentary to the review of materials discussed thus far, or the chatbot can be a standalone feature via which users can seek information and answers to specific questions related to regulatory and enforcement issues and topics. In one example embodiment, the chatbot implements ML algorithm 140 and, as such, the various training and operational features discussed above with respect to FIGS. 1-3C apply equally to the chatbot implemented by ML algorithm 140.

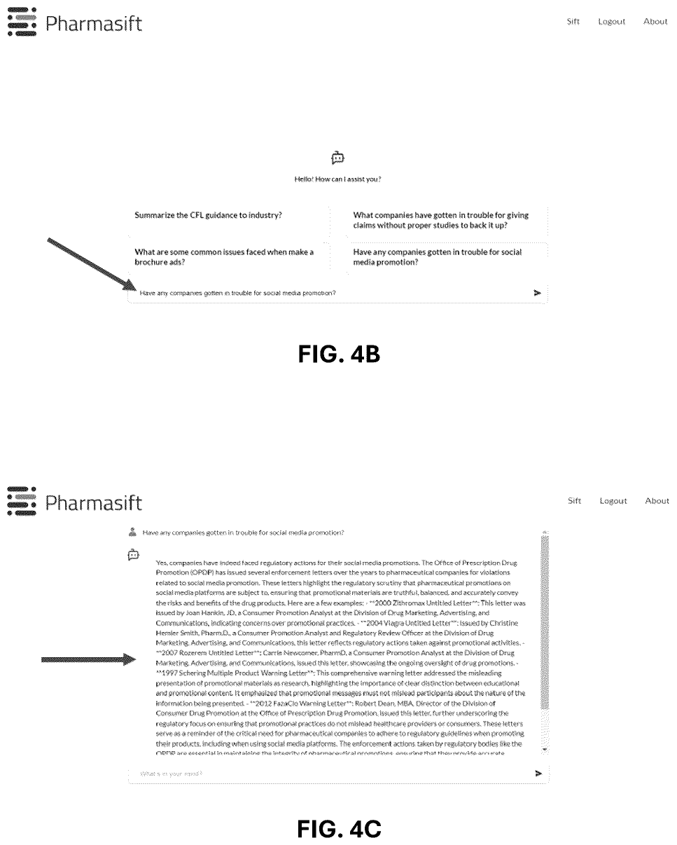

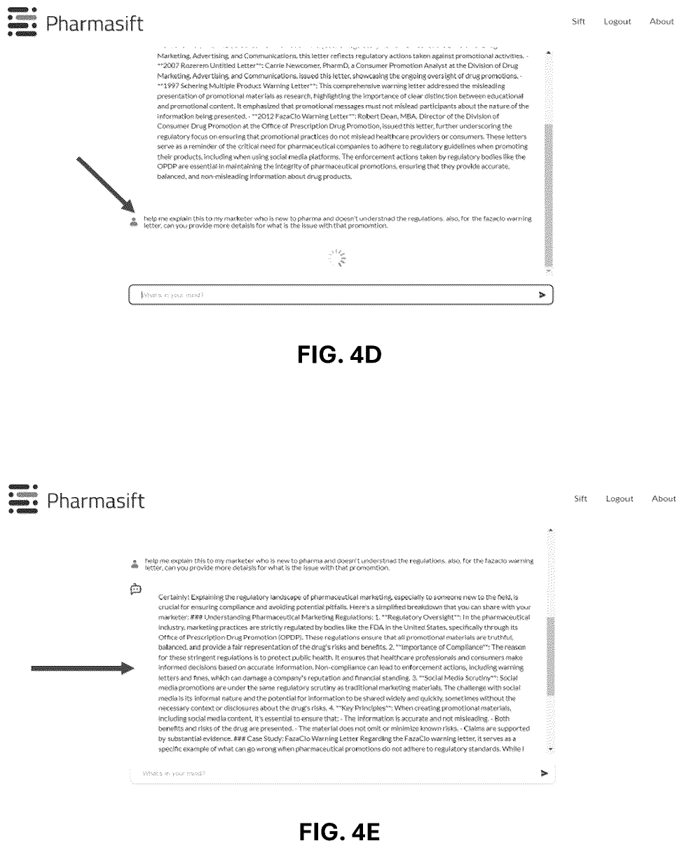

An example chatbot user interface is depicted in FIGS. 4A-4E. In FIG. 4A, a query field is shown in which a user can enter a specific question. Frequently asked questions, or most recently asked questions from a particular user, also can be included as one-click options for ease of use.

In FIG. 4B, a question has been entered into the query field: “Have any companies gotten in trouble for social media promotion?” A helpful feature of the chatbot implemented by ML algorithm 140 is its ability to handle natural language queries. As such, a user is not required to know industry terminology, technical jargon, or even spell words correctly in order to interact with the chatbot. Furthermore, the chatbot feature can be used as a follow-on to output 180 if a user wishes to gather more information by asking specific questions related to content provided by M L algorithm 140 in output 180.

In FIG. 4C, ML algorithm 140 has provided an answer to the query. In this example, ML algorithm 140 provides specific examples by name so that a user has context and information for further review. In some embodiment, the output of chatbot provided by ML algorithm 140 can include excerpts clipped from documents, hyperlinks to referenced documents or other sources, photos, or other output that could be useful or helpful in order for a user to process and understand the answer.

In FIG. 4D, a user has asked the chatbot a follow-up question: “Help me explain this to my marketer who is new to pharma and doesn't understand the regulations, also, for the fazaclo warning letter, can you provide more details for what is the issue with that promotion.” This follow up query actually contains two separate requests of the chatbot (ML algorithm 140): 1) “Help me explain this to my marketer who is new to pharma and doesn't understand the regulations,” and 2) “for the fazaclo warning letter, can you provide more details for what is the issue with that promotion.”

As can be seen in the example screenshot of FIG. 4E, the chatbot feature of ML algorithm 140 is able to handle this more complex query with ease. A gain, the output of chatbot provided by ML algorithm 140 here also can include excerpts clipped from documents, hyperlinks to referenced documents or other sources, photos, or other output that could be useful or helpful in order for a user to process and understand the answer.

In some embodiments, the chat feature enables users to communicate with data, and this communication can be implemented in various ways. In one embodiment, the chat feature is a chatbot agent equipped with several tools to query the database(s). An example chatbot agent 510 is depicted in FIG. 5.

Whenever a user makes a query (e.g., via the query field depicted in FIGS. 4A-4E), chat agent 510 automatically decides to use the most appropriate tool, such as enforcement letters tool 520, CFR guidelines tool 522, or others. The tool 520, 522 further generates SQL queries to search for the relevant information in an SQL database, which may be part of a system database 530.

There are some cases in which chat agent 510 gets no relevant data from querying the database(s) 530 directly. To handle such cases, semantic searching can be implemented in one embodiment. To accomplish this, embeddings of all the documents are created and stored in a vector store or database 532. Whenever chat agent 510 queries vector database 532, agent 510 receives in return all content that is semantically similar to the user query. Once chat agent 510 gets the relevant data from the SQL database (system database 530) or vector database 532, agent 510 generates its response and responds to the user.

To enable chat agent 510 to semantically search data regarding CFR guidelines, FDA enforcement letters, and other relevant (and, as discussed herein above, curated) data, embeddings of all the available data on these and other selected sources can be created. In one embodiment, contextual embedding chunks can be created (such as using Spacy Text Splitter), such that text is broken into meaningful chunks. These chunks can serve a better meaning of the text and are more likely to be adopted while applying semantic search with the user chat query. These chunks are then converted into embeddings and can be stored in vector database 532.

Chat agent 510 is provided with multiple data sources via tools (e.g., 520, 522) and can decide which tool to use to search for the appropriate answer based on the user query. Once decided, chat agent 510 calls the tool for searching into system database 530, where raw text is available for all the documents. If system database 530 fails to return any relevant content, agent 510 applies to embed on the user query and search for the contextual similarity in vector database 532.

Chat agent 510 is also equipped with a session-based memory 540, which can provide a more user-friendly chat experience. For example, user messages and AI-generated responses can be stored in the SQL database, which can then be used by chatbot 510 to better understand the user question and to keep the context.

Additionally, users can upload PDFs and other document types documents and query chat agent 510 based thereupon. In one embodiment, any uploaded documents are stored (e.g., in memory 130) and processed, such as by using a GPT-40 vision model as an Optical Character Recognition (OCR) to extract textual as well as images and tables data.

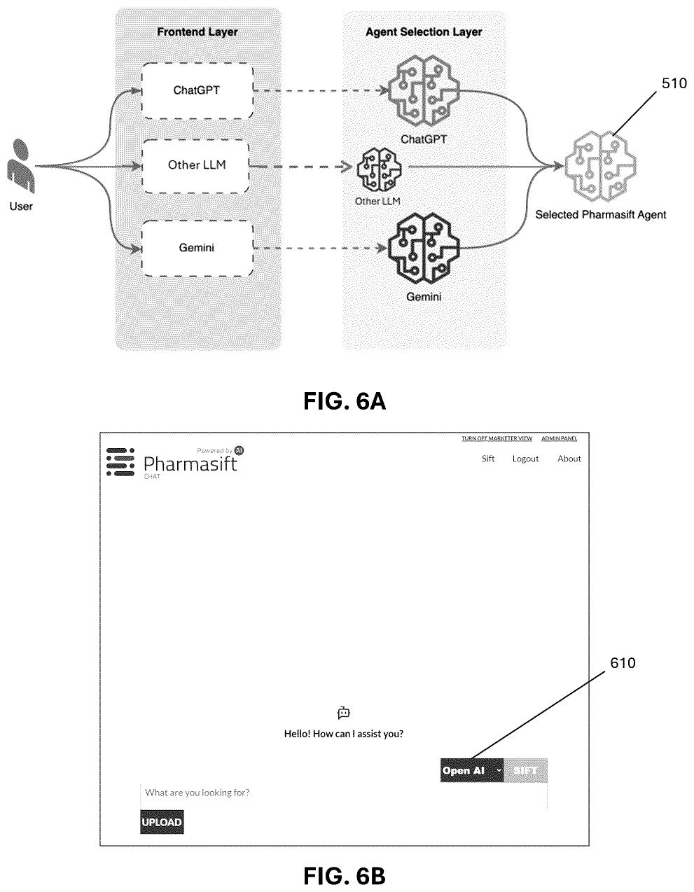

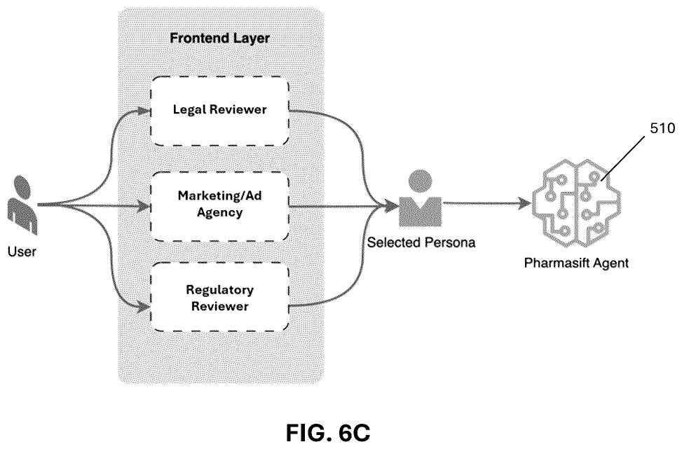

Other available features of embodiments of chat agent 510 are AI model and agent persona selection. Referring also to FIGS. 6A and 6B, a user may also choose an AI model, from among (including but not limited to) ChatGPT, Gemini, and other large language models (LLMs). Referring also to FIG. 6C, a user may additionally choose a persona, selected from among (including but not limited to) a legal reviewer persona, a regulator reviewer persona, a marketing reviewer persona, an advertising agency persona, and others. In general, each available model and persona thereof has the same power of responding to user chat questions based on relevant data and saving the chat history in memory, though memory 540 is shared among agents 510. For example, if in a session a user starts a chat with Gemini, and after a few messages desires to switch to GPT, the GPT agent will take over with context of the user's chat with the previous model. To facilitate model selection, a selection drop-down option 610 is provided in the chat user interface.

Users also have the option of choosing a chat agent persona of chat agent 510. Personas can vary based on functional scope, such as regulatory, legal, or marketing, or according to other characteristics. Generally speaking, personas may be developed according to any function, task, or characteristic of any one or any process involved in the development, review, and approval of promotional and other relevant materials. This can enable chat agent 510 to respond to user chat queries with information that is most relevant to the selected persona, provided in terminology or at level most suited for a selected persona, or otherwise tailored.

Therefore, disclosed herein are various embodiments of systems and methods for reviewing and analyzing information related to products, services, or industries subject to regulation to check whether the information is in compliance or may be subject to enforcement action. Embodiments of these systems and methods can include and implement one or more specially trained ML algorithms. The special training can include feeding the ML algorithm with training data that includes proprietary information including or related to legal analysis, guideline interpretation, industry guidance, and other specially developed information, as well as selective publicly available information such as government laws or rules, enforcement action letters or other materials, approved information, and other information that is or could be relevant to the review and analysis of the subject information. The trained ML algorithm then can review materials provided to the system or answer specific questions asked of the chatbot feature.

Though examples discussed herein primarily relate to life sciences industries, including those regulated by the FDA like in particular drugs and pharmaceuticals, embodiments can have applicability to other regulated industries, such as healthcare, petroleum/oil/gas and coal, electric power, motor vehicles, finance, air travel and transportation, natural resources such as fishing and game, and ocean and fresh water transportation. These are only examples, however, and embodiments of the ML algorithm and chatbot can be used in almost any setting in which it is helpful to specially train the algorithm with expert and proprietary information in order to improve the efficacy and elevate the performance of the ML algorithm.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

It should be understood that the individual operations used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. An artificial intelligence (AI)-powered regulatory review system comprising:
a data set stored in memory and comprising at least one of life sciences-related advertising and promotion rules or regulations, wherein at least a portion of the data set is proprietary and not publicly available; and
at least one processor communicatively coupled with the memory to execute code to:
receive electronic material for review via a user interface, the electronic material for review comprising proposed regulated content that includes at least one of text, an image, or a video,
analyze the electronic material for review by:
applying at least one filter to the electronic material for review to create filtered content;
applying a large language model (LLM) trained on the data set and at least one jurisdiction-specific regulatory framework data set to the filtered content to generate a compliance analysis based on the data set, and
provide the compliance analysis as an output report with which a user can selectively interact via a user interface, the output report comprising at least a first portion of the compliance analysis related to the data set and a second portion of the compliance analysis related to the at least one jurisdiction-specific regulatory framework data set.

2. The system of claim 1, wherein the LLM is configured to:
evaluate individual content modules in the filtered content for compliance with life sciences regulatory requirements; and
assemble the filtered content into a final suggested promotional piece based at least in part on the evaluating.

3. The system of claim 2, wherein the LLM is configured to tag each individual content module with a regulatory risk score and provide a citation mapping to the life sciences regulatory requirements.

4. The system of claim 2, wherein regulatory risk levels and suggested edits are provided as part of the output report.

5. The system of claim 1, wherein the compliance analysis comprises historical interactions, compliance flags, and reviewer notes, and wherein the compliance analysis is stored and retrievable to enable tracking of rationales and precedents for proposed regulated promotional content-related decisions.

6. The system of claim 5, wherein the LLM is configured to analyze reviewer behavior and outcomes over time to additionally generate suggested best practices and improve review consistency.

7. The system of claim 1, wherein the LLM is configured to update the compliance analysis based on prior reviewer approvals, rejections, and comments related to life sciences materials.

8. The system of claim 1, wherein the life sciences-related advertising and promotion rules or regulations relate to at least one of a pharmaceutical, a drug, or a medical device.

9. A method for assisting in review and approval of life sciences content, the method comprising:
receiving proposed life sciences content via a user interface;
parsing the received proposed life sciences content to identify any of claims, references, or risk-benefit language;
comparing the identified claims, references, or risk-benefit language to a database of at least one of jurisdiction-specific life sciences rules or regulations;
providing the identified claims, references, or risk-benefit language to a large language model trained on a data set comprising at least one of life sciences-related advertising and promotion rules or regulations, wherein at least a portion of the data set is proprietary and not publicly available;
based on results of the comparing and the providing, generating suggested modifications to the proposed life sciences content to enhance compliance with the at least one of jurisdiction-specific life sciences rules or regulations; and
provide the suggested modifications as an output report with which a user can selectively interact via the user interface.

10. The method of claim 9, wherein the database includes regulations from at least one governmental agency or regulatory entity.

11. The method of claim 9, wherein generating suggested modification comprises identifying at least one portion of the identified claims, references, or risk-benefit language likely to be considered to violate the at least one of jurisdiction-specific life sciences rules or regulations, or to result in an unsatisfied rule.

12. The method of claim 11, wherein the at least one portion of the identified claims, references, or risk-benefit language likely to be considered to violate the at least one of jurisdiction-specific life sciences rules or regulations includes an unsupported superiority claim, an omission or minimization of risk information, an overstatement of efficacy, a false or misleading presentation of efficacy or safety, a broadening of an indication, or a preapproval promotion.

13. The method of claim 9, wherein the life sciences content relates to at least one of a pharmaceutical, a drug, or a medical device.

14. A method for providing regulatory guidance using a conversational artificial intelligence system, comprising:
training a large language model (LLM) a data set comprising at least one of life sciences-related advertising and promotion rules or regulations, wherein at least a portion of the data set is proprietary and not publicly available, and at least one jurisdiction-specific regulatory framework data set;
receiving, via a user interface, a user-submitted query about a life sciences claim;
analyzing the user-submitted query by the trained LLM; and
outputting via the user interface a result of the analyzing from the LLM referencing at least one of specific regulatory guidance documents, precedent enforcement actions, and product-specific labeling, or proprietary rules based on at least one of the data set or the at least one jurisdiction-specific regulatory framework data set.

15. The method of claim 14, wherein outputting the result of the analyzing further comprises providing suggested compliant claim alternatives to the user-submitted query generated by the LLM based on product labeling and prior internal or external review decisions in the data set.

16. The method of claim 14, wherein outputting the result of the analyzing further comprises including recommendations tailored based on at least one of a functional role of the user or historical interaction behavior.

17. The method of claim 14, wherein the user-submitted query comprises at least one of text input or a file input, and wherein the user interface is configured to provide the result of the analyzing from the LLM comprising at least one of regulatory feedback, a suggested compliant alternative, or a relevant search result from a regulatory database.

18. The method of claim 14, wherein outputting the result of the analyzing further comprises providing at least one of a hyperlink citation to a relevant section of a product labels, a regulatory guidance document, or an enforcement action.

19. The method of claim 14, wherein the life sciences advertising and promotion rules or regulations relate to at least one of a pharmaceutical, a drug, or a medical device.

20. A method comprising:
training a large language model (LLM) on a data set comprising life sciences advertising and promotion regulations, wherein at least a portion of the data set is proprietary and not publicly available;
receiving a user-submitted query comprising any of a natural language question about regulated content or a related claim, proposed regulated content as text, or proposed regulated content as a file; and
filtering the user-submitted query to create filtered content;
providing the filtered content to the trained LLM;
providing a response from the trained LLM to the user-submitted query, based on the filtered content, referencing at least one of specific regulatory guidance documents, precedent enforcement actions, product-specific labeling, and proprietary rules based on the data set.

21. The method of claim 20, wherein the LLM is configured to consider user-specific data.

22. The method of claim 20, wherein the life sciences advertising and promotion regulations relate to at least one of a pharmaceutical, a drug, or a medical device.

23. The system of claim 1, wherein the electronic material for review further comprises supplemental documents, and wherein the LLM is trained based on the supplemental documents.
